(19) United States
(12) Patent Application Publication
(10) Pub. No.: US 2025/0135169 A1
(43) Pub. Date: May 1, 2025

(54) PATIENT-SPECIFIC IMPLANT AND METHOD FOR ITS MANUFACTURE

(71) Applicants: MEDIZINISCHE HOCHSCHULE HANNOVER, Hannover (DE); HÖRSYS GMBH, Hannover (DE)

(72) Inventors: Verena SCHEPER, Suthfeld (DE); Theodor DOLL, Isernhagen (DE); Farnaz MATIN, Hannover (DE); Samuel JOHN, Hannover (DE); Thomas LENARZ, Isernhagen (DE)

(21) Appl. No.: 18/689,551

(22) PCT Filed: Nov. 18, 2022
(86) PCT No.: PCT/EP2022/082441

§ 371 Date: Mar. 6, 2024

(30) Foreign Application Priority Data
Nov. 22, 2021 (EP) 21209553.3

(51) Int. Cl. A61M 31/00; B33Y 50/00; B33Y 80/00

(52) U.S. Cl. CPC A61M 31/002; B33Y 50/00; B33Y 80/00; A61M 2205/0238; A61M 2205/04; A61M 2205/583; A61M 2207/00; A61M 2209/04; A61M 2210/0668

(57) ABSTRACT

The invention relates to a patient-specific implant (5) for insertion into a body cavity of a patient, for example into the round window niche of the middle ear. The invention also relates to a method of manufacturing such an implant (5).

The invention relates to a patient-specific implant for insertion into a body cavity of a patient, for example into the round window niche of the middle ear, or in another body cavity like:
inner ear,
cochlea,
vestibular system,
cranium,
cavum tympani,
mastoid,
sinus frontalis.

The invention also relates to a method of manufacturing such an implant.

There are already proposals for the manufacture of patient-specific implants. However, the suitability of such known implants for insertion into a patient's body cavity is severely limited, especially if the body cavity is accessible only through a narrowed body opening, such as the round window niche.

It is an object of the invention to provide an improved implant which can be inserted simply and reliably even in body cavities which are accessible only via a narrowed body opening. Furthermore, an advantageous method for manufacturing such an implant is to be disclosed.

This object is achieved by a patient-specific implant for insertion into a body cavity of a patient, the implant having an implant body and a handle attached directly or indirectly to the implant body, wherein the handle is adapted for holding and guiding the implant during insertion into the body cavity, the implant body having an outer contour which corresponds predominantly to the inner contour of the body cavity of the patient, characterized in that at least the implant body contains a medical active substance which is deliverable from the implant body to the patient. Such an implant can be placed reliably and safely in the body cavity. The implant can be held by the handle and guided by the user or a robot during the insertion process. At the implantation site, the implant body can then deliver the medical agent to the patient.

Advantageously, the implant is designed for permanently remaining in the body cavity, at least for a duration required for the delivery of the medical agent to the patient. In other words, the implant is designed for permanently remaining in the body cavity for this duration with the handle, which means the handle is not detached from the implant body after insertion of the implant in the body cavity. For example, the handle may be permanently connected to the implant body such that the handle is not detachable from the implant body. Of course, the handle should be small enough that it can remain in the body cavity without causing problems for the patient. In particular, the maximum dimension of the handle can be smaller than the maximum dimension of the implant body. For the insertion of the implant for insertion into the body cavity a tool, e.g. tweezers or a forceps, can be used which is removed after the insertion process. In particular, the handle can have a size which does not allow insertion of the implant in the body cavity without an additional tool which is not part of the implant.

The patient-specific implant can be patient-specific in one or more of the following ways:
The patient-specific implant can be a customized implant which size and shape are adapted to the geometrical and other requirements of an individual patient,
the patient-specific implant can be a customized implant where the medical active substance is selected for treatment of an individual patient,
the patient-specific implant can be a customized implant which is adapted for implantation to an individual patient.

According to an advantageous embodiment of the invention, the implant body is formed as a solid body having substantially no air pockets. This has the advantage that the implant body can be provided both mechanically stable and to a certain extent compressible for the insertion process.

According to an advantageous embodiment of the invention, the implant body can have a higher compressibility than the handle. This has the advantage that the implant can be reliably held and guided via the more rigid handle compared to the implant body, while the implant body can also be guided through constricted areas to the body cavity due to its higher compressibility. For example, the implant body may be provided from a material having a higher compression modulus than the material of the handle. It is also possible that the implant body and the handle both have the same material and same compressibility.

According to an advantageous embodiment of the invention, the implant body is elastically compressible by at least 20%. In this way, the implant body can have sufficient softness when inserted into the body cavity. For example, the implant body can be compressed to at least 20% less volume than in the uncompressed state without permanent damage.

However, the implant body does not necessarily need to be compressible, to change in volume, but in shape. For example, the implant body can be sqeezed to be put through narrow passages. For this purpose, the implant body can have an elasticity and/or flexibilty such it can be sqeezed during the insertion process. According to an advantageous embodiment of the invention, the implant body can have a greater elasticity than the handle. This allows the implant body to conform well to the body cavity and, during the insertion process, to the possibly narrower body caves upstream of the body cavity. For example, the implant body may be made of a material with a greater modulus of elasticity than the material of the handle. It is also possible that the implant body and the handle both have the same material and same elasticity.

According to an advantageous embodiment of the invention, the implant can have a marking by which the position and/or orientation of the implant during the implanting process in the body cavity is recognizable to the user. This has the advantage that the position and/or orientation of the implant can be checked by the user at any time during the implantation process and the correct implantation direction can be ensured. Such a marking thus serves as an orientation aid for the user when implanting the implant into the body cavity. The marking may, be in the form of an asymmetric shape of the handle itself, for example the handle may be shaped like an arrow. It is advantageous if the marking is arranged on the handle, because it is then clearly visible to the user during the entire implantation process.

According to an advantageous embodiment of the invention, the implant body is biodegradable. Accordingly, the implant body may slowly decompose in the body cavity, which has the advantage that no additional intervention is required for explantation of the implant body. The entire implant may also be biodegradable.

According to an advantageous embodiment of the invention, the implant body can have a multilayer structure of active substance-loaded material with active substance concentrations and/or active substances differing in the layers. In this way, a time-programmed active substance release can be realized optionally with respect to the active ingredient concentration and/or with respect to the type of active ingredient.

According to an advantageous embodiment of the invention, active substance-producing cells are integrated in the implant body, by means of which the medical active substance that can be delivered to the patient can be produced. In this way, a constant delivery of the active substance to the patient can be ensured over a longer period of time.

According to an advantageous embodiment of the invention, at least the implant body is manufactured by means of an additive manufacturing process. This allows a particularly precise patient-specific provision of the implant body or the implant. The entire implant may also be manufactured using an additive manufacturing process. The additive manufacturing process can be, for example, a 3D printing process, e.g. laser printing, extrusion printing, inkjet printing.

The object of the invention is further achieved by a method for manufacturing a patient-specific implant of the type explained above, comprising the following steps:
a) Reading e.g. three-dimensional patient data into a computer program, the patient data showing the shape of the body cavity into which the implant is to be placed.
b) Modeling of the implant body in the computer program, e.g. by segmenting the anatomic structures that surround the body cavity, according to the shape of the body cavity using the patient data, for example with a model based approach or with a threshold based approach.
c) Adding a handle to the implant body in the computer program, e.g. based on one or more pre-defined geometries. The user can orient (rotate) the handle such that it is most suitable for the implantation procedure. For example the tip of the handle can be made to point towards a landmark that is visible in situ during the surgery. The computer programs closes the potential gap between the handle and the filled shape of the body cavity.
d) Exporting the data characterising the implant modeled in the computer program in a format suitable for an additive manufacturing device, optionally removing potential undercuts that would complicate additive manufacturing.

The object of the invention is further achieved by a method for manufacturing a patient-specific implant of the type explained above, comprising the following steps:
a) Reading three-dimensional patient data into a computer program, the patient data showing the shape of the body cavity into which the implant is to be placed.
b) Segmenting the anatomic structures that surround the body cavity, for example with a model based approach or with a threshold based approach.
c) Modeling of the implant body in the computer program by filling the body cavity up to the border of the individual anatomic segmentations. As the body cavity often does not necessarily have a clearly defined all-surrounding border, the automatic filling starts at the point deepest inside the cavity and the lumen is filled outwards until it “overflows”. This overflow is detected when the filled volume increases rapidly due to filling the space outside the narrowed entry of the body cavity. Additionally, the user of the computer program can manually limit the filling.
d) Adding a handle to the implant body in the computer program based on one or more pre-defined geometries. The user can orient (rotate) the handle such that it is most suitable for the implantation procedure. For example the tip of the handle can be made to point towards a landmark that is visible in situ during the surgery.

The computer programs closes the potential gap between the handle and the filled shape of the body cavity.
e) Exporting the data characterising the implant modeled in the computer program in a format suitable for an additive manufacturing device, optionally removing potential undercuts that would complicate additive manufacturing.

The previously explained advantages can also be realized by this method. The patient data can be obtained, for example, by means of an examination process of the patient performed before the execution of the method, e.g. by a radiological three-dimensional examination, in particular by an examination by means of computer tomography, magnetic resonance tomography, time-of-flight laser scanning, 3D microscopy, 3D reconstruction based on image processing techniques, OCT imaging, and/or digital volume tomography.

The invention is explained in more detail below with reference to exemplary embodiments using drawings.

### THE DRAWINGS SHOW IN

FIG. 1 a schematic representation of part of the inner ear of a patient,

FIG. 2 a patient-specific implant,

FIG. 3 a marking element,

FIG. 4 a manufacturing process of the implant.

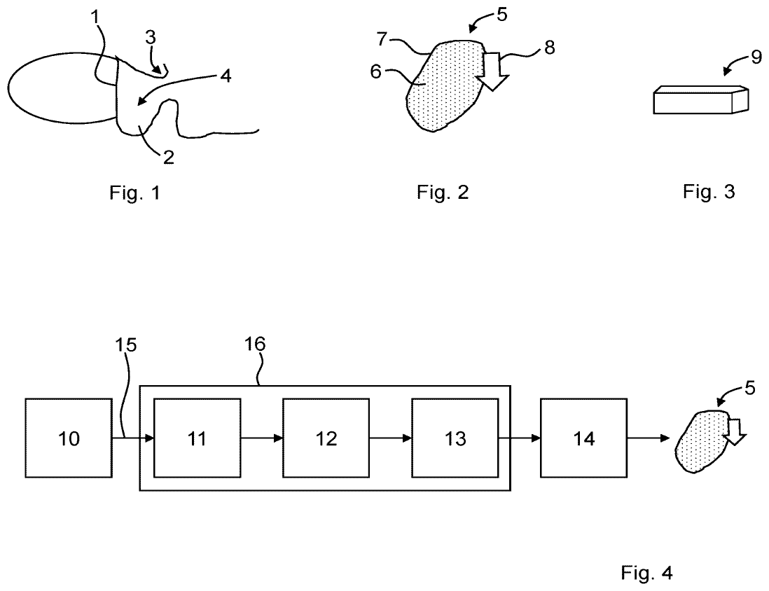

FIG. 1 shows a schematic representation of an area of the inner ear of a living organism, e.g. a human or an animal. The round window niche 2 can be seen, which is separated from other areas of the inner ear, in particular the Scala tympani, by the round window membrane 1. If an implant is to be placed in the round window niche 2, it must first be passed through the isthmus 3 of the round window niche 2 according to the arrow 4, i.e. a constricted area, before it can be placed in the larger round window niche 2. For this purpose, it is advantageous if the implant or at least the implant body has sufficient compressibility and/or flexibility so that it can be guided through the isthmus 3 without damaging the patient. After passing the isthmus 3, the implant body expands again and fits into the round window niche 2.

FIG. 2 shows a patient-specific implant 5 designed for insertion into the round window niche 2. The patient-specific implant 5 has an implant body 7 and a handle 8 attached to the implant body 7. The handle 8 can be used for holding and guiding the implant body 7 during the insertion process. The handle 8 may have a marking as an orientation aid for the user or may be designed in the form of such a marking, for example in the form of an arrow. At least one medical agent is present in the implant body 7, which can be delivered to the patient by the implant body 7 arranged in the round window niche 2, for example by diffusing the medical agent through the round window membrane 1 into the scala tympani of the inner ear.

FIG. 3 shows an advantageous design of a marking element 9 which can be arranged on the implant 5 as an orientation aid, for example on the handle 8. The marking element 9 can also form the handle 8 at the same time. The shape of the marking element 9 can be designed as a cuboid, with one side being pointed, in order to obtain information about the orientation of the implant.

FIG. 4 shows the manufacturing process of the implant 5 by computer aided manufacturing. Prior to the actual manufacturing process, patient data are determined in a step 10, e.g. by means of an X-ray three-dimensional examination. The patient data 15 are read into a computer program 16. Within this computer program 16, various steps are performed. In a step 11, a modeling of the implant body 7 is performed, for example by selecting and segmenting in the patient data 15 the relevant region of the body cavity into which the implant 5 is to be inserted. In a subsequent step 12, a handle for handling by the surgeon is added to the implant body modelled in the computer program. Based on this, in a step 13, the corresponding print output file for an additive manufacturing device can be generated, for example an STL file. This print file is then output from the computer program 16 to an additive manufacturing device 14, by which the real implant 5 is then produced by means of additive manufacturing.

The invention describes a new class of implants that can be inserted as a solid body into the round window niche (RWN), cochlea, vestibular system, cranium, cavum tympani, mastoid or sinus frontalis and can locally deliver active substances for the treatment of various diseases. For atraumatic placement, secure retention and effective drug delivery, it is advantageous to customize the implants to the geometry of the individual target region. In order to be able to be inserted into the cavity, it is advantageous if the implant is compressible and/or flexible and has a marking for the correct implantation direction.

The implant according to the invention may thus having one, more or all of the following features:
Full body
Individual shape and size specific to the patient
at the same time mechanically stable and compressible and/or flexible for insertion
medical active substance containing and releasing
Marking for orientation
Grab handle

To avoid explantation of the implants, they can be made of biodegradable materials. Targeted drug release into e.g. the inner ear may also be implemented. Additionally, the implant according to the invention may have one, more or all of the following features:
1) degradable
2) induced dissolution or acceleration of degradation.
3) Target tissue-oriented medical active substance release
4) time-programmed release of the medical active substance by means of a layer-by-layer structure of active ingredient-laden material with layer-by-layer different active ingredient concentrations or different active ingredients.
5) Integration of drug-producing cells

Round window niche implants allow the treatment of the inner ear with active substances that diffuse from the implant through the round window membrane into the inner ear. Thus, pathologies such as Menier's disease, hearing loss and cochlear implant-associated trauma reactions can be treated. To date, it is not possible to diffuse drugs into the inner ear in a controlled manner, since the drug-releasing implant surface is not adapted to the round window membrane of the individual patient. There are considerable variations in size and shape, as well as a pseudomembrane if necessary. The implant of the invention is the first drug-releasing system that is adapted to the individual anatomy of the patient and can therefore be safely implanted in the round window niche to deliver its active ingredients to the inner ear in an effective and controlled manner. Its compressibility allows atraumatic insertion through the isthmus into the round window niche, where it remains, as the expansion after insertion prevents it from slipping out.

The handle may have an arrow shape and thus also serves to orient the surgeon in which direction the implant is to be placed. The shape and thickness of the handle can be varied so that an optimal support structure for the individualized implant body can be printed.

In a manufacturing step, the handle for the implant is created on the surface of the implant facing the middle ear (step 12 in FIG. 4). The shape of the handle can be chosen as a cuboid with one side pointed to provide information about the orientation of the implant. By default, this side points in the direction of the basal rotation of the cochlea at the round window to allow the surgeon to implement the planned fit in the niche. The orientation and dimensions of the handle can be adjusted by the user, while the software ensures that there is no overlap with bony structures of the middle ear, based on the specified threshold. The dimensions of the handle can be selected to be small enough that, if necessary, only the arrow mark is imaged and no more handle is required. Accordingly, the implant can subsequently be printed with or without a handle, but with has a guide for the surgeon.

In general, biopolymers, synthetic polymers, polymer blends, nanocomposites, functional polymers and cell-loaded systems can be used as materials for the implant body and/or the handle. The materials to be printed should be dimensionally stable on the one hand, but on the other hand compressible in order to be introduced into the cavity. Hydrogels are suitable for this purpose, as they have a lattice structure in the linked state, which enables the inclusion of active substances with subsequent diffusion out of the material. At the same time, the material should be degradable in order to avoid explantation of the implant after successful therapy.

### Suitable Materials are for Example

Silicone
Alginate
Cellulose
Chitosan
Polyethylene glycol (PEG)
   and their combinations with each other as well as with other non-flexible materials such as
   Poly (lactic acid) (PLA), Poly (glycolic acid) (PGA), Poly (lactic-co-glycolic acid) (PLGA), Poly (caprolactone) (PCL), Poly (amide, Poly (anhydride), Poly (phosphazene), Poly (dioxanone)

The materials are either mixed with medical active substances prior to manufacture or the implant is filled with medical active substances by diffusion after manufacture.

The amount of medical active substances to be introduced depends on the pathology addressed and varies accordingly.

Some possible medical active substances are listed in Table 1 below:

TABLE 1
Exemplary drug classes and active ingredients.

| | Immuno- |
|---|---|
| Anti-inflammatory | suppressants |
| Dexamethasone, dexamathasone sodium phosphate | Mitomycin |
| Triamcinolone, Methylprednisolone, Prednisolut | Sirolimus |
| Mometasone Fuorate | |
| Fluticasone proprionate, Etanercept, Diclofenac | |
| Antioxidant | Antibacterial |
| Butylhydroxyltoluene, Edaravon | Ciprofloxacin |
| H3 antihistamine | Neuroprotective |
| Betahistine | BDNF, GDNF, NT- |
| | 3, IGF, NTF, FGF |
| | and agonists of |
| | the corresponding |
| | receptors |

The implant can be manufactured, for example, by means of additive manufacturing processes listed below.

### 3.3.1 Laser Printing:

Stereolithography (SLA)
two-photon polymerization (2PP)
laser-induced forward transfer

### 3.3.2 Extrusion Printing

Fused deposition modeling (FDM)
direct ink writing

### 3.3.3 Inkjet Printing

Selective laser sintering (SLS)
Digital light processing (DLP) technology

The implants should be as biodegradable as possible=bioresorbable=bioabsorbable. They should either dissolve independently in the body or be induced to dissolve.

Reduced dissolution of alginate can be done by injection of alginate lyase into the middle ear. Chitosan can be dissolved by injection of a low pH solution. It must be noted that the solutions must be biocompatible.

Immortalized, drug-producing cells can be introduced into the implant. The factors to be produced include, but are not limited to, growth factors and other proteins, as well as antioxidant molecules, which can be neuroprotective as well as antioxidant and thus protect specific inner ear structures from degeneration. The cells should survive in the implant for at least 4 weeks. The drugs must diffuse from the implant into the inner ear, but the cells must be firmly entrapped in the material to avoid an immune response from the patient. Cells of any origin such as stem cells, fibroblasts or human umbilical vein endothelial cells (HUVEC) can be introduced.

1. A patient-specific implant for insertion into a body cavity of a patient, comprising:
an implant body; and
a handle attached directly or indirectly to the implant body, wherein the handle is adapted for holding the implant during insertion into the body cavity,
wherein the implant body comprises an outer contour which corresponds predominantly to an inner contour of the body cavity of the patient,
wherein at least the implant body contains a medical active substance which is deliverable from the implant body to the patient.

2. The implant according to claim 1, wherein the implant body is formed as a solid body having substantially no air pockets.

3. The implant according to claim 1 wherein the implant body has a same or a higher compressibility than the handle.

4. The implant according to claim 1 wherein the implant body is elastically compressible by at least 20%.

5. The implant according to claim 1 wherein the implant body has a same or a greater elasticity than the handle.

6. The implant according to claim 1 wherein the handle is wider than the implant body in an extent predominantly orthogonal to the insertion direction in order to function as a depth stop for avoiding too deep over-insertion of the implant.

7. The implant according to claim 1 wherein the handle is shaped such that it rests in a form-fit at on a constriction at an entry of the body cavity when the implant body reaches a correct implantation depth and a correct position in the body cavity.

8. The implant according to claim 1 further comprising a marking by which a spatial position and/or orientation of the implant during implanting in the body cavity is recognizable to a user.

9. The implant according to claim 8, wherein the marking is arranged on the handle.

10. The implant according to claim 1 wherein the handle is formed as a position and/or orientation aid which supports a user in guiding the implant during insertion into the body cavity.

11. The implant according to claim 1 wherein the implant body and/or the handle is biodegradable.

12. The implant according to claim 1 wherein the implant body has a multilayer structure of medical active substance-loaded material with medical active substance concentrations and/or medical active substances differing in the layers of the multilayer structure.

13. The implant according to claim 1 wherein the implant body comprises active substance-producing cells integrated in the implant body, wherein the medical active substance that can be delivered to the patient is produced by the active substance-producing cells.

14. The implant according to claim 1 wherein the implant is adapted for insertion into a round window niche of an ear of a patient.

15. The implant according to claim 1 wherein at least the implant body is manufactured by an additive manufacturing process.

16. A method of manufacturing a patient-specific implant according to claim 1, comprising:
a) reading patient data into a computer program encoded on a non-transient medium, the patient data describing a shape of a body cavity into which the implant is to be placed,
b) modeling of an implant body in the computer program according to the shape of the body cavity and optionally a shape of a constriction at an entry of the body cavity using the patient data,
c) adding a handle to the implant body in the computer program and optionally modeling a shape of the handle using the patient data to match shape of the constriction at the entry of the body cavity in the computer program, and
d) outputting data characterizing the implant modelled in the computer program to an additive manufacturing device.
